(19) United States
(12) Patent Application Publication
(10) Pub. No.: US 2020/0352621 A1
(43) Pub. Date: Nov. 12, 2020

(54) THERMAL RESISTANCE HEATER

(71) Applicant: Xueli YU, Yiwu City, Zhejiang Province (CN)

(72) Inventor: Xueli YU, Yiwu City, Zhejiang Province (CN)

(21) Appl. No.: 16/640,698

(22) PCT Filed: Jun. 5, 2019
(86) PCT No.: PCT/CN2019/090204

§ 371 Date: Feb. 20, 2020

(30) Foreign Application Priority Data
Jun. 6, 2018 (CN) 201810573096.9

(51) Int. Cl. A61B 18/08; H05B 1/02; H05B 3/10; A61F 7/00

(52) U.S. Cl. CPC A61B 18/08; H05B 1/025; A61B 2018/00577; A61F 7/007; H05B 3/10

(57) ABSTRACT

A thermal resistance heater with a thermal conductive shell that is used to contact with tumor tissues and conduct heat therefor is provided. A thermal resistance is disposed inside the thermal conductive shell and is self-heated via current. The thermal energy is converted from the electrical energy according to Joule's Law. The heater includes a heat radiator disposed inside the shell and is used to disperse the heat generated by the thermal resistance and conduct the heat to the shell evenly. A thermal-conduction compensation arm and the heat radiator are contacted for achieving that a temperature of the shell is the same with a specific place or an error there-between is within a threshold. A temperature sensor is used to obtain a surface average temperature of the conductive shell by collecting temperatures over the thermal-conduction compensation arm. By adjusting position the temperature sensor is disposed on thermal-conduction compensation arm, the temperature sensed by the temperature sensor can be the same with a surface temperature of a heating zone of the shell or an error there-between is within a threshold. It achieves that a controller precisely controls a surface temperature of heater.

### FIELD OF THE DISCLOSURE

The disclosure is generally related to a thermal resistance heater, and more particularly to a thermal resistance heater applied for treatment of tumor.

### BACKGROUND OF THE DISCLOSURE

The clinical equipment for tumor hyperthermia is such as a radio-frequency hyperthermia device or an electromagnetic wave hyperthermia device for tumor treatment. The conventional equipment heats the tumor tissue with electromagnetic waves. The principle is that the human tissues will be heated by absorbing the electromagnetic waves. The human tissues convert the absorbed electromagnetic waves to heat. The electric-thermal conversion is not only related to the intensity of electromagnetic waves, but also to structure and dielectric constant of the human tissues. Since the tumor tissue is a combination of multiple structures, the temperatures heated by the radio frequency or electromagnetic waves over the entire tumor issue are uneven, and the temperature differences inside the tumor are large. Therefore, it may appear that the tumor is not inactivated completely due to even though a portion of the tumor tissue has been carbonized but another portion of the tumor tissue does not reach the temperature for inactivation.

In the process of tumor hyperthermia, it is important that the thermal resistance heater is controlled to increase the temperature of the tumor tissue precisely. Otherwise, the tumor cannot be inactivated completely or controlled to death. Furthermore, the heat may harm the normal tissues or organs of the patient.

Two important functions of the thermal resistance heater are to produce thermal energy but not electromagnetic radiation, and to control the surface temperature of the heater. Therefore, it is necessary for the thermal resistance heater to dispose a temperature sensor. However, the position where the temperature sensor is disposed on the thermal resistance heater affects an accuracy of temperature measurement. If the temperature sensor is disposed on a surface of the thermal resistance heater and directly contacts with the tumor tissue, some problems may occur as follows.

Firstly, the temperature sensor and the thermal resistance heater are point-contacted and the temperature measured by the temperature sensor is only the temperature of a point of the thermal resistance heater but not an average value of the entire heater. Secondly, if the point to be measured by the temperature sensor is at a region with poor heat-dissipation, the temperature of the region with poor heat-dissipation is higher than other regions with good heat-dissipation since the heat of the point may not be transferred efficiently. Therefore, the temperature, e.g. an average temperature, over the entire heater may be lower than the required therapeutic temperature and affect the effect of heat treatment.

### SUMMARY OF THE DISCLOSURE

The disclosure is generally related to a thermal resistance heater. The technical program to be solved by this thermal resistance heater is to precisely control a surface temperature of the heater for providing a safe and an effective for tumor hyperthermia.

For achieving the above purposes, a solution for hyperthermia for tumor is provided as follows.

A thermal resistance heater is provided for the hyperthermia. The thermal resistance heater is used to heat the tumor tissue so as to inactivate and ablate the tumor tissue. The thermal resistance heater includes a conductive shell that is used to contact with the tumor tissue for conducting the heat.

A thermal resistance can be self-heated via a current that flows through the thermal conductive shell.

A heat radiator is disposed inside the thermal conductive shell and is used to disperse the heat generated by the thermal resistance and conduct the heat to the thermal conductive shell evenly.

A thermal-conduction compensation arm contacts with the heat radiator for achieving that the temperature of a specific position is the same with the thermal conductive shell or an error there-between is required to be within a threshold.

A temperature sensor obtains an average temperature of the surface of the thermal resistance heater with the conductive shell by collecting temperatures at the specific position of the thermal-conduction compensation arm.

Further, the thermal resistance heather also includes a controller. The controller adjusts a current flowing the thermal resistance according to temperatures collected by the temperature sensor so as to stabilize temperature signals of the temperature sensor to a preset value. Therefore, a surface temperature of the thermal conductive shell can be precisely controlled.

Still further, the temperature signals of the temperature sensor are transmitted to the controller via a temperature sensor wire (5). The current outputted by the controller is transferred to the thermal resistance via a thermal resistance wire.

Further, temperature of an end of the thermal-conduction compensation arm that contacts with the heat radiator is high, and temperature of the other end of the thermal-conduction compensation arm that is away from the heat radiator is low. By adjusting a position of the temperature sensor disposed on the thermal-conduction compensation arm, a thermal conductive distance can be adjusted for achieving a purpose of temperature compensation and finally confirming the specific position where the temperature sensor is disposed.

Further, the temperature sensor and the thermal resistance form a one-piece structure through the heat radiator and the thermal-conduction compensation arm.

Still further, the thermal conductive shell is preferably a stainless steel shell, and/or the heat radiator is preferably a heat-dissipation copper core (2).

Further, an outer wall of the heat radiator seamlessly contacts with an inner wall of the thermal conductive shell. The inner wall of the heat radiator thermally contacts with the thermal resistance.

The thermal resistance heater is configured to provide following benefits.

(1) The temperature sensed by the temperature sensor can be configured to be the same with a surface temperature of the heater, or allow an error there-between within a threshold by adjusting a fixed position where the temperature sensor disposed on the thermal-conduction compensation arm. Therefore, the controller can precisely control the surface temperature of heater.

(2) In the disclosure, the tumor tissue is inactivated and ablated by heating the thermal resistance. The controller can precisely control the surface temperature when the temperature sensor disposed on the thermal-conduction compensation arm is used to sense the temperature. The procedure for inactivation and apoptosis of tumor can therefore be controlled for preventing the patient's normal tissues or organs from being destroyed.

(3) Since the tumor hyperthermia of the disclosure adopts a thermal resistance to heat the tumor tissue via a means of thermal conduction, the internal temperatures of the tumor tissue appear to be a gradient distribution. According to the gradient distribution of the temperatures, the temperature of the tissue near a thermal resistance heater is high and the temperature of the tissue being away from the thermal resistance heater is low. There is no exception to any tumor tissue structure. When a temperature value of a thermometer that is placed at an intersectional area between the tumor tissue and normal tissue reaches a valid temperature, it indicates that the temperatures of the entire tumor tissue are not lower than the valid temperature. Therefore, when the temperatures generated by thermometer 2 placed at the intersectional area between the tumor tissue and the normal tissue can be stabilized within 43° C. to 45° C. for a while, the method for tumor hyperthermia can completely inactivate the tumor tissue without harming the normal human tissue.

(4) The data generated by the temperature-controllable thermal resistance heater and the thermometer is real, reliable and continuous since the thermal resistance heater of the disclosure does not produce electromagnetic radiation and also not interfere the temperature sensor.

(5) A PID temperature-control circuit can be used in the thermal resistance heater of the disclosure. The PID temperature-control circuit makes the temperature-controllable thermal resistance heater 1 generating stable and precise data. Therefore, in the process of controlling temperature, it achieves that the tumor hyperthermia has minimized overshoot, precise temperature control, and safe and reliable hyperthermia procedure.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram depicting an axial sectional structure of a thermal resistance heater according to one embodiment of the disclosure;

FIG. 2 shows a schematic diagram depicting a temperature distribution of a thermal-conduction compensation arm in one example of the disclosure;

FIG. 3 is a schematic diagram depicting components of a device for hyperthermia for tumor according to one embodiment of the disclosure;

FIG. 4 is a schematic diagram depicting the thermal resistance heater and a thermometer according to first embodiment of the disclosure;

FIG. 5 is a schematic diagram depicting the thermal resistance heater and a thermometer according to second embodiment of the disclosure;

FIG. 6 is a schematic diagram depicting the thermal resistance heater and a thermometer according to third embodiment of the disclosure;

FIG. 7 is a diagram showing a function for controlling temperature in a process of controlling temperature according to one embodiment of the disclosure; and

FIG. 8 shows a structural diagram of a thermometer in one embodiment of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The embodiments of the disclosure are described with references to FIG. 1 to FIG. 8 as follows.

A thermal resistance is adopted in a heater of the disclosure for heating and conducting the heat to tumor tissue. The temperatures inside the tumor tissue appear a gradient distribution. The distribution of temperatures over the tumor tissue shows that the temperature of the tissue near the thermal resistance heater is high and the temperature of the tissue being away from the thermal resistance heater is low. There is no exception to any tumor tissue structure. The thermal resistance heater heats the tumor tissue. The temperature of the entire tumor tissue should be larger than an inactivation temperature if the temperature of an edge of the tumor tissue reaches the inactivation temperature. Therefore, the entire tumor tissue can be inactivated thoroughly.

The heater of the disclosure may not generate electromagnetic radiation since the heater uses a thermal resistance. Therefore, the thermal resistance heater does not interfere with the nearby electronic devices or electronic components.

Reference is made to FIG. 1, which shows that a thermal resistance wire 4 is linked to a controller, and a temperature sensor wire 5 is also linked to the controller. A heating current being outputted by the controller is transferred to a thermal resistance 3 via the thermal resistance wire 4. The thermal resistance 3 generates heat by being energized with the heating current. The heat is then conducted to raise temperature of a heat-dissipation copper core 2. The heated heat-dissipation copper core 2 then heats a thin-walled stainless steel shell 1 by means of thermal conductance.

In the meantime, the temperature of the heat-dissipation copper core 2 can be conducted to a temperature sensor 6 via a thermal-conduction compensation arm 7. Signals relating to the temperature sensed by the temperature sensor 6 are transmitted to the controller via the temperature sensor wire 5. The controller then adjusts the heating current according to the signals provided by the temperature sensor 6 and therefore causes the temperature signals to be stabilized at a preset value. This process achieves that the surface temperature of the thermal conductive shell can be precisely controlled.

The surface temperature of the stainless steel shell 1 is much lower than the temperature of the heat-dissipation copper core 2 because the stainless steel has a low thermal conductivity. If the temperature sensor 6 is simply placed onto the heat-dissipation copper core 2, the temperature measured by the temperature sensor 6 may be very different from an actual temperature of surface of the stainless steel shell 1. However, the temperature sensor 6 may collect wrong temperature signals when the temperature sensor 6 contacts with the region with poor heat-dissipation of the stainless steel shell 1 if the temperature sensor 6 is directly mounted on the stainless steel shell 1.

For the temperature sensor 6 disposed on a heat-dissipation copper core can reveal an average temperature of the surface of the stainless steel shell 1 precisely, the temperature sensor 6 can be disposed on the thermal-conduction compensation arm 7. The position where the temperature sensor 6 is disposed on the thermal-conduction compensation arm can be adjusted for regulating a thermal conductive distance so as to compensate the temperature of the heater. Accordingly, the surface temperature of the stainless steel shell 1 can be the same with the temperature sensed by the temperature sensor 6, or an error there-between occurs but is within a tolerance range.

In an aspect of the disclosure, by adjusting a fixed position where the temperature sensor is disposed on the thermal-conduction compensation arm, the temperature sensed by the temperature sensor is the same with the surface temperature of the thermal conductive shell, or an error existed between the temperatures can be controlled to be within a tolerance range. The controller can therefore controls the surface temperature of the thermal conductive shell precisely.

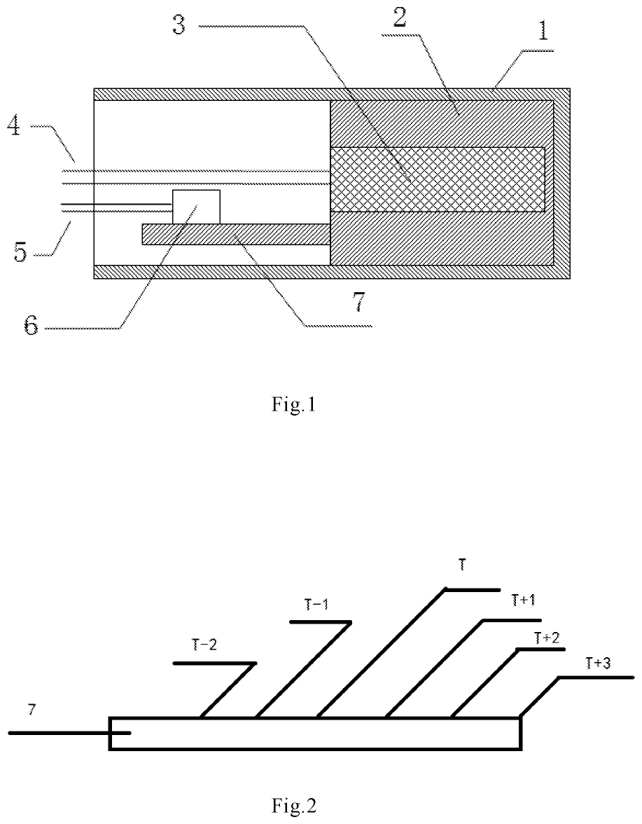

As shown in FIG. 2, if the surface of the stainless steel shell 1 has an average temperature ‘T’, the temperature of the portion where a right side of a thermal-conduction compensation arm 7 is connected with a heat-dissipation copper core 2 is required to be higher than the average surface temperature of the stainless steel shell 1. However, the temperature of the other end of the thermal-conduction compensation arm 7 being distanced from the connection portion will be gradually decreased. For example, the thermal-conduction compensation arm 7 has six measurement points, and the temperatures of these six measurement points being measured from left to right are labeled with ‘T−2’, ‘T−1’, ‘T’, ‘T+1’, ‘T+2’ and ‘T+3’. The temperature sensor 6 may be required to be disposed at the measurement point with temperature ‘T’ for actually revealing or extremely approaching the average temperature of the stainless steel shell 1.

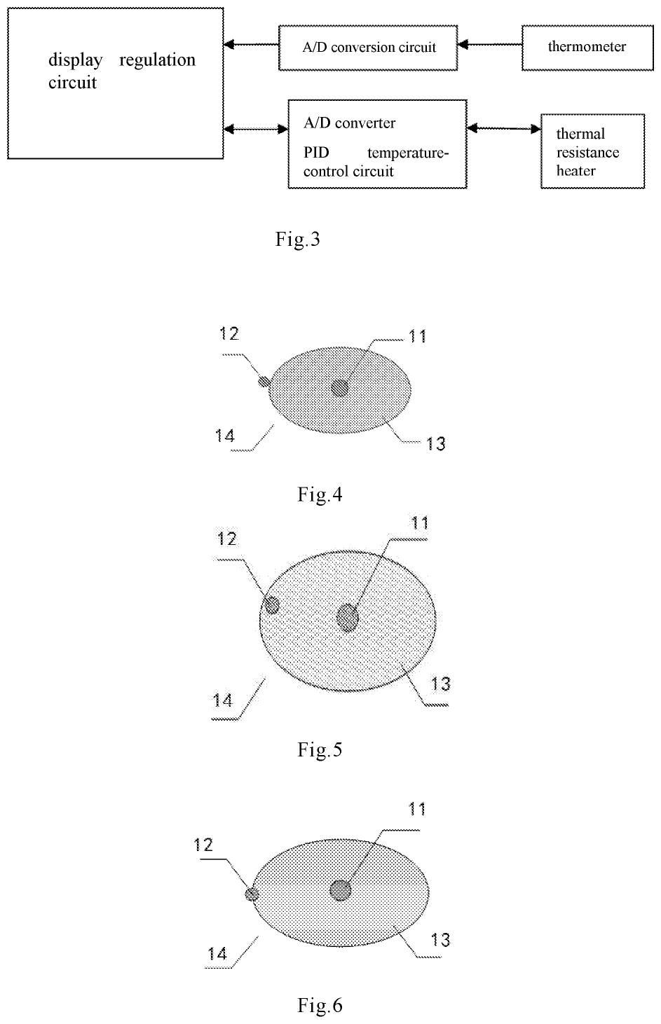

FIG. 3 is a schematic diagram depicting a tumor hyperthermia device that includes a thermal resistance heater 11. The thermal resistance heater 11 is placed in a central region of a tumor and configured to conduct heat to the tumor through a thermal resistance. In the meantime, the temperature of the self-heated thermal resistance heater 11 can be controlled precisely. A thermometer 12 is placed at an intersectional area between the tumor tissue and normal tissue for measuring temperature. The controller is connected with the thermal resistance heater 11 so as to display and control the heating temperature. The controller is also connected with the thermometer 12 for displaying the temperature measured by the thermometer 12.

The thermal resistance heater 11 is used to heat the tumor tissue in order to inactivate or ablate the tumor tissue. A thermal conductive shell is included in the thermal resistance heater 11 and contacted with the tumor tissue for conducting heat into the tumor tissue. The thermal resistance is disposed inside the thermal conductive shell and is self-heated via current. The heater includes a heat radiator that is disposed inside the thermal conductive shell and is used to disperse the heat generated by the thermal resistance, especially to conduct the heat to the thermal conductive shell evenly. The heater includes a thermal-conduction compensation arm that is connected with the heat radiator. The thermal-conduction compensation art causes the temperature at a specific position to be the same with the temperature of the thermal conductive shell, or have an error there-between within a range. The temperature sensor is used to collect the temperatures at a specific position of the thermal-conduction compensation arm so as to calculate an average temperature of the thermal conductive shell.

The temperature signals generated by the temperature sensor are transmitted to the controller via the temperature sensor wire 5, and the current outputted by the controller is transferred to the thermal resistance via the thermal resistance wire. In which, the controller regulates the current flowing through the thermal resistance according to the temperature signals generated by the temperature sensor. The temperature signals can thus be stabilized at a present value. The purpose of precisely controlling the surface temperature of thermal conductive shell can be achieved.

Temperature of an end of the thermal-conduction compensation arm that contacts with the heat radiator is high. Otherwise, temperature of the other end of the thermal-conduction compensation arm that is away from the heat radiator is low. By adjusting a position where the temperature sensor is disposed on the thermal-conduction compensation arm, a thermal conductive distance can be adjusted. Therefore, a purpose of temperature compensation can be achieved and the specific position where the temperature sensor is disposed can be finally confirmed.

The temperature sensor and the thermal resistance form a one-piece structure through the heat radiator and the thermal-conduction compensation arm. The thermal conductive shell is a stainless steel shell and/or the heat radiator is a heat-dissipation copper core 2. An outer wall of the heat radiator seamlessly contacts with an inner wall of the thermal conductive shell. An inner wall of the heat radiator thermally contacts with the thermal resistance.

The controller includes a display regulation circuit, an A/D converter, and a PID temperature-control circuit. The display regulation circuit is used to display a heating temperature of the thermal resistance heater 11 and a measurement temperature of the thermometer 12. The heating temperature of the thermal resistance heater 11 is adjustable. The A/D converter is used to convert the analog signals such as the temperature signals generated by the thermal resistance heater 11 and thermometer 12 into digital signals. The PID temperature-control circuit is used to control the thermal resistance heater 11 for providing an accurate and stable heating temperature. It should be noted that PID temperature-control circuit controls the heating temperature by a proportional calculation, an integral calculation or a differential calculation.

An operational principle of the tumor hyperthermia device of the disclosure is described as follows.

Step 1: the thermal resistance heater 11 is placed at a central region of the tumor tissue 13, and the thermometer 12 is placed at the intersectional area between the tumor tissue 13 and the normal tissue 14.

Step 2: when the tumor hyperthermia device enters a heating state, the thermal resistance heater 11 continuously heats the tumor tissue 3 according to a preset temperature. The analog signals generated by the thermometer 12 are converted to the digital signals by an A/D conversion circuit. The digital signals are then transmitted to the display regulation circuit for displaying the measurement temperature of the thermometer 12. Similarly, the analog signals generated by the thermal resistance heater 11 are also converted to the digital signals by the A/D converter, and the digital signals are then transmitted to the display regulation circuit for displaying the heating temperature of the thermal resistance heater 11. The PID temperature-control circuit controls a heating current according to a difference between an actual temperature of the thermal resistance heater 11 and a preset temperature automatically. Therefore, the heating temperature of the thermal resistance heater 11 can be stabilized to a preset value precisely.

Step 3: an operational temperature of the thermal resistance heater 11 can be regulated to a stable value, i.e. the preset value, according to another difference between an actual temperature measured by the thermometer 12 and a demand value. This aspect causes the temperature measured by the thermometer 12 to be stabilized to the demand value. The temperature heated by the thermal resistance heater 11 can be kept for a period of time so as to inactivate and ablate the tumor tissue.

FIG. 4 is a schematic diagram depicting the temperature-controlled thermal resistance heater 11 that is placed in a central region of the tumor tissue 13. The thermometer 12 is placed at an intersectional region between the tumor tissue 13 and the normal tissue 14. For example, the thermometer 12 is configured to contact with the normal tissue 14.

In FIG. 5, the temperature-controlled thermal resistance heater 11 is placed at the central region of the tumor tissue 13. The thermometer 12 is placed at an intersectional region between the tumor tissue 13 and the normal tissue 14. For example, the thermometer 12 contacts with the tumor tissue 3.

Reference is made to FIG. 6. The thermal resistance heater shown in the diagram is placed at a central region of the tumor tissue 13. The thermometer 2 is placed at the intersectional region between the tumor tissue 13 and the normal tissue 14. For example, the thermometer 12 contacts with the tumor tissue 13 and the normal tissue 14.

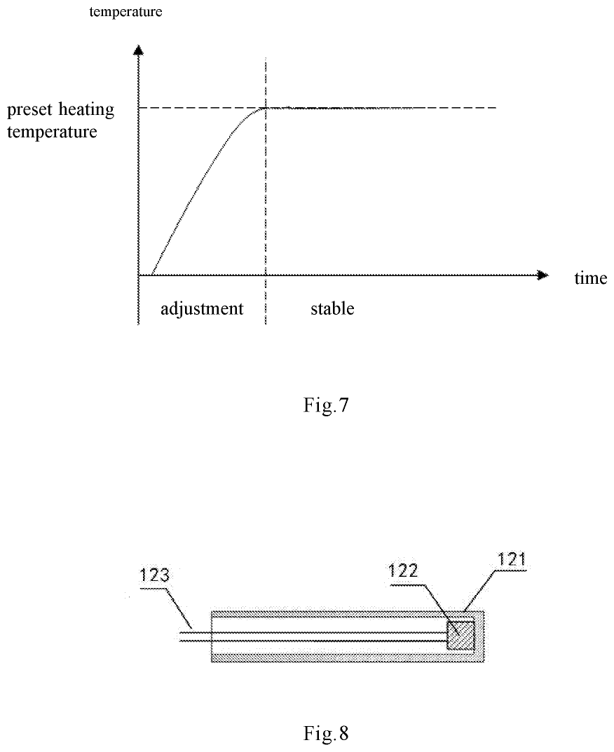

In FIG. 7, the characteristic diagram shows that the PID temperature-control circuit makes the temperature of the thermal resistance heater 11 to be stabilized and with a high precision. There is a very small overshoot during temperature regulation. The temperature can be controlled precisely. Therefore, the process of hyperthermia is safe and reliable.

FIG. 8 schematically shows a structural diagram of a thermometer in one embodiment of the disclosure. The thermometer 12 includes a shell 121, a temperature sensor 122 and a temperature sensor wire 123. The temperature sensor 122 contacts with an inner wall of a lowermost end of the sensor shell 121. The temperature sensor 122 connects with the temperature sensor wire 123 and the controller. The temperature sensor 122 of the thermometer 12 is a platinum resistance, a thermocouple, or a thermistor.

The tumor hyperthermia device of the disclosure has the following beneficial effects as compared with a conventional tumor hyperthermia device.

Firstly, the thermal resistance heater uses a thermal resistance to heat the tumor tissue by means of thermal conduction. The internal temperatures of the tumor tissue appear to be a gradient distribution. The gradient distribution of the temperature indicates that the temperature of the tissue near the thermal resistance heater 11 is high, but the temperature of the tissue away from the heater 11 is low. There is no exception to any structure of tumor tissue. The temperature of the thermometer 2 denotes that the temperatures of the entire tumor tissue is not lower than the temperature measured by the thermometer 2 when the thermometer is placed at the intersectional region between the tumor tissue and the normal tissue.

In an exemplary example, the thermometer 2 is placed at the intersectional region between the tumor tissue and the normal tissue. The tumor tissue can be inactivated without harming the normal tissue if the temperature of the thermometer 2 remains stable between 43° C. and 45° C. for a period of time. An improved tumor hyperthermia method is implemented.

Secondly, there is no electromagnetic radiation when the thermal resistance heater of the disclosure uses the thermal resistance to heat the tumor tissue. The temperature sensor won't be interfered. Therefore, the temperature of the thermal resistance heater and the thermometer is actual, reliable and continuous.

Thirdly, since the thermal resistance heater adopts the PID temperature-control circuit, the thermal resistance heater 1 provides a stable and a high-precision temperature. There is a very small overshoot in the process of temperature regulation, the temperature can be controlled precisely, and therefore the process of hyperthermia is safe and reliable.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A thermal resistance heater, used to heat a tumor tissue for inactivating and ablating the tumor tissue, comprising:
a thermal conductive shell used to contact with the tumor tissue for conducting heat inside the tumor tissue;
a thermal resistance disposed inside the thermal conductive shell and self-heated via a current;
a heat radiator disposed inside the thermal conductive shell for dispersing the heat generated by thermal resistance and conducting the heat to the thermal conductive shell evenly;
a thermal-conduction compensation arm contacted with the heat radiator for allowing a temperature of a specific position of the thermal-conduction compensation arm to be the same with the thermal conductive shell, or an error there-between to be within a threshold; and
a temperature sensor used to obtain an average temperature of the thermal conductive shell by collecting temperatures of the specific position of the thermal-conduction compensation arm.

2. The thermal resistance heater according to claim 1, further comprising a controller that adjusts the current flowing the thermal resistance according to temperatures collected by the temperature sensor so as to stabilize temperature signals of the temperature sensor to a preset value for precisely controlling a surface temperature of the thermal conductive shell.

3. The thermal resistance heater according to claim 1, wherein the temperature signals of the temperature sensor are transmitted to the controller via a temperature sensor wire (5), and the current outputted by the controller is transferred to the thermal resistance via a thermal resistance wire.

4. The thermal resistance heater according to claim 1, wherein the temperature of an end of the thermal-conduction compensation arm that contacts with the heat radiator is high, and temperature of the other end of the thermal-conduction compensation arm that is away from the heat radiator is low; by adjusting a position of the temperature sensor disposed on the thermal-conduction compensation arm to adjust a thermal conductive distance, a purpose of temperature compensation is achieved and the specific position where the temperature sensor is disposed is confirmed.

5. The thermal resistance heater according to claim 4, wherein the temperature sensor and the thermal resistance form a one-piece structure through the heat radiator and the thermal-conduction compensation arm.

6. The thermal resistance heater according to claim 4, wherein the thermal conductive shell is a stainless steel shell (1) and/or the heat radiator is a heat-dissipation copper core (2).

7. The thermal resistance heater according to claim 1, wherein, an outer surface of the heat radiator seamlessly contacts with an inner wall of the thermal conductive shell, and an inner wall of the heat radiator thermally contacts with the thermal resistance.
